(19) United States
(12) Patent Application Publication
(10) Pub. No.: US 2019/0080043 A1
(43) Pub. Date: Mar. 14, 2019

(54) METHOD AND DEVICE FOR MODELLING HUMAN OR ANIMAL TISSUE

(71) Applicants: Optimo Medical AG, Biel (CH); Universität Bern Verwaltungsdirektion, Bern (CH)

(72) Inventors: Philippe Büchler, Gossens (CH); Elena Businaro, Nidau (CH); Harald P. Studer, Liebefeld (CH)

(73) Assignees: Optimo Medical AG, Biel (CH); Universität Bern Verwaltungsdirektion, Bern (CH)

(21) Appl. No.: 15/773,602

(22) PCT Filed: Oct. 25, 2016
(86) PCT No.: PCT/EP2016/075676

§ 371 Date: May 4, 2018

(30) Foreign Application Priority Data
Nov. 5, 2015 (CH) 1606/15

(51) Int. Cl. G16H 50/50; G16H 20/40; A61B 3/00

(52) U.S. Cl. CPC G06F 19/12; G16H 50/50; G06F 19/28; A61B 3/0025; G16H 20/40

(57) ABSTRACT

Disclosed is a computer-implemented method for modelling human or animal patient tissue. The method includes: acquiring a stressed patient topography data set, the stressed patient topography data set describing a topography of at least one patient tissue surface under mechanical stress; determining an input data set; evaluating a statistic model for the input data set, thereby obtaining an output data set; determining, from the output data set, a relaxed patient topography data set. The statistic model includes a set of Gaussian Processes and is defined by a pre-determined model parameter set, the model parameter set including at least one Gaussian Process parameter for each Gaussian Process, and wherein the statistic model is independent from the patient tissue to be modelled. Disclosed is further a computerized device for carrying out such method. Disclosed are further a computer-implemented method and a computerized device for determining a parameter set.

### CROSS-REFERENCE TO RELATED APPLICATION

This application is a 371 National Stage application of International Application No. PCT/EP2016/075676, filed on Oct. 25, 2016, which claims foreign priority to Swiss (CH) Application Serial No. 1606/15 filed on Nov. 5, 2015, the contents of which are incorporated herein by reference in their entireties.

### BACKGROUND OF THE INVENTION

### Field of the Invention

The present invention relates the field of methods and devices for simulating human or animal tissue, in particular in the context of surgery simulation and surgery planning. The present invention further relates to methods and devices for determining required parameter sets for such simulations. A particular filed of application is the simulation and planning of corneal surgeries.

### Description of the Prior Art

A variety of visual defects and visual problems, such as myopia (short-sightedness), hyperopia (far-sightedness) or astigmatism are corrected according to the state of the art via surgical interventions at the eye (corneal surgery). The refractive correction of the eye is largely carried out via ophthalmologic laser systems which cut and/or remove eye tissue, thus modifying the refractive properties. Alternatively or additionally to ophthalmologic laser systems, scalpels may be used are used.

For the planning and general simulation of corneal surgeries, patient-specific finite element models have been proposed. In order to carry out such simulations, the corneal topography is first determined via ophthalmologic measurement devices and a patient-specific finite element model is subsequently computed based on the measured data.

### BRIEF SUMMARY OF THE INVENTION

A major problem that is associated with this kind of simulation is that the associated medical images and data are acquired in-vivo and therefore relate to the tissue in its physiological situation, which is frequently under mechanical stress. This is typically the case for soft tissue such as ligaments, arteries and in particular the human eye. For the human eye, the mechanical stress is associated with the intraocular pressure. However, the level of initial mechanical stress or strain in the tissue cannot be directly measured on the patient. Biomechanical models, such as finite element models as mentioned before, in contrast, require a relaxed configuration.

It is therefore required to determine the relaxed (i. e. stress-free) configuration from the acquired configuration under stress or—equivalently—to determine the initial stress that is present in the tissue during data acquisition, in particular during topography determination.

Several methods have been investigated and proposed for this purpose. A common drawback of all of such methods is a high computational effort that is required for the numeric computations.

Computation time, however, is an important issue for the applicability and acceptance in the daily clinical routine. Therefore, the relaxed configuration of the patient tissues should be obtained as efficiently as possible.

It is an overall object of the present invention to provide methods and devices that allow the determination of a relaxed tissue configuration under typical clinical circumstances. In comparison to the state of the art, the required computational effort for determining the relaxed tissue configuration is favourably reduced.

The overall object is generally achieved by the subject of the independent claims. Particularly favourable as well as further exemplary embodiments are further defined by the subject of the dependent claims as well as the overall disclosure of the present document.

The present invention is based on the major insight that the relation between the stressed and relaxed configuration of the patient tissue and in particular the relation between the stressed and the relaxed surface topography can be described, in the clinical praxis, by means of a statistic model with a number of Gaussian Processes. This statistic model can be pre-determined or “trained” in advance and is independent from the specific patient. Once the statistic model is defined, the evaluation of the statistic model and accordingly the determination of the relaxed tissue surface geometry is computed faster as compared to the state of the art. Depending on the computational equipment, the computation may, in some embodiments, be carried out within seconds or fractions of seconds. An explicit determination of the relaxes configuration according to the state of the art, in contrast, takes up t about 20 min with typical state-of-the-art computational equipment.

According to an aspect, the overall object is achieved by providing a computer-implemented method for modelling human or animal patient tissue for the simulation of a surgical intervention. The method includes acquiring a stressed patient topography data set, the stressed patient topography data set describing a topography of at least one patient tissue surface under mechanical stress. The method further includes determining, from the stressed patient topography data set, an input data set. The method further includes evaluating a statistic model for the input data set, thereby obtaining an output data set. The method further includes determining, from the output data set, a relaxed patient topography data set, the relaxed patient topography data set describing a topography of the at least one patient tissue surface in absence of mechanical stress. The statistic model is defined by a pre-determined model parameter set, the model parameter set including at least one Gaussian Process parameter for each Gaussian Process. The model parameter set is independent from the patient tissue to be modelled. That is, while the statistic model is evaluated individually for each patient tissue, the statistic model as such and its model parameters are independent from the specific patient tissue and may accordingly be pre-determined.

The particularly low computational effort and the corresponding short time that is required for the computation for determining the relaxed patient topography data set is caused by the fact that the determination of the relaxed tissue configuration from an acquired stressed tissue configuration is replaced by a pre-determined statistic model that can be evaluated quickly. The Gaussian Processes are each defined by at least one Gaussian Process parameter.

The method and its specific and exemplary embodiments as further described in the following may especially be carried out with a computerized device as also described in the following.

The step of acquiring the stressed patient topography data set may be carried out via medical measurement devices, such as ophthalmologic measurement devices. In dependence of the specific application and the patient tissue to be modelled, however, the topography data set may also be acquired from other data sources, such as medical imaging data, e.g. ultrasonic imaging data, X-ray imaging data, topography data, an the like. Furthermore, the data set may be present in a computer memory or be red in from a data carrier, such as a CD-ROM. The data source and the way of acquiring the data is accordingly not decisive.

This document mostly refers to tissue of the human eye, in particular corneal tissue, as patient tissue to be modelled. It is to be understood, however, that the same methods and devices may be applied to other tissues, in particular soft tissues, such as alignments or arteries. References to eye tissue in specific examples and exemplary embodiments are therefore to be equally understood as references to tissue in general. It is assumed, however, that the reference tissues as will be described further below and the patient tissue to be modelled are of generally the same type and show identical mechanical behaviour. If, for example, the patient tissue to be modelled to is corneal tissue, the reference tissues are corneal tissues as well.

Within this document, the topography of a tissue surface refers to the distance/height of any point given of the tissue surface from a reference surface, such as a reference plane, as measured, e.g. in millimetres or micrometers. For corneal tissue or generally eye tissue, for example the surface topography is defined by the height of any surface point over a reference plane, the reference plane being perpendicular to the optical axis. In this document, the term “topography” generally refers to tissue surface topographies within the before-described meaning.

The phase “stressed tissue” refers to a tissue as present under in-vivo conditions. The phrase “relaxed” tissue” refers to a fully relaxed tissue without internal mechanical stress. The phrase “statistic model” refers to a set of Gaussian Processes.

As will be described further below in more detail, data sets may be particularly be organized particularly as vectors and/or matrices, with each vector or matrix element representing a single value. Therefore, references to vectors and/or matrices are also references to corresponding data sets.

In an embodiment, the input data set is a stressed patient decomposition coefficient set, the stressed patient decomposition coefficient set being obtained via a parametric decomposition from the stressed patient topography data set, the stressed patient decomposition coefficient set forming the input data set for the statistic model. Here, the output data set is a relaxed patient decomposition coefficient set. The statistic model defines a relation between coefficients of the stressed patient decomposition coefficient set and the relaxed patient decomposition coefficient set, respectively.

The parametric decomposition is a (theoretically infinite) series in a finite approximation, i. e. of finite order). Each element of the series is defined by at least one decomposition coefficient. The different parametric decompositions that are employed in the method, are in some embodiments of identical type and order. In such embodiments, they accordingly have identical numbers of decomposition coefficients. As mentioned before in the general context of data sets, coefficient sets may be organized as matrices and/or vectors. However, the coefficients and coefficient sets may also be arranged in other ways.

Specific examples of suited parametric decompositions are discussed in more detail further below.

In an embodiment, the step of determining the relaxed patient decomposition coefficient set includes evaluating a relation between the coefficients of the stressed patient decomposition coefficient set, the coefficients of a pre-determined stressed reference decomposition coefficient set and the coefficients of a pre-determined relaxed reference decomposition coefficient set.

Here, the relaxed patient decomposition coefficient set and the stressed reference decomposition coefficient set each comprise the decomposition coefficients for a set of reference tissues, for example a set of reference corneas. Exemplarily, the reference decomposition coefficient sets are arranged as matrixes where, for example, the decomposition coefficients of the different reference tissues are arranged in different columns, while the single decomposition coefficients for the same reference tissue are arranged in different columns. Further, the relaxed patient decomposition coefficient set and the stressed patient decomposition coefficient set may be arranged as vectors with each vector element comprising a decomposition coefficient.

Since the reference decomposition coefficient sets are pre-determined, they are readily available when doing the computation for patient tissue. They may be an integral part of a computer program product, such as the computer code, or be provided as coefficient file or the like. The reference decomposition coefficient sets may especially be determined using a device and method for determining the parameters set as described further below in more detail.

In an embodiment, the step of determining the relaxed patient decomposition coefficient set includes evaluating a set of covariances between coefficients of the stressed patient decomposition coefficient set and coefficients of the stressed reference decomposition coefficient set. The set of covariances may for example be arranged as a covariance matrix.

In an embodiment, the at least one Gaussian Process parameter includes, for each Gaussian Process, a tuple of a variance parameter and a scaling parameter. The tuples may, for example be arranged in Gaussian Process parameter matrix. The tuples of variance parameters may make or be part of the pre-determined model parameter set. As will be explained in more detail further below in the context of exemplary embodiments, statistic uncertainty may optionally be considered. In this case, a noise variance parameter is added for each Gaussian Process, resulting in a Gaussian parameter triple for each Gaussian Process.

The Gaussian Process parameters may especially be used for determining the before-described covariance matrix.

Since the Gaussian Process parameters are pre-determined, they are, like the reference decomposition coefficient sets, readily available when doing the computation for patient tissue. They may also be an integral part of a computer program product, such as a computer code, or be provided as coefficient file or the like. The Gaussian Process Parameters may also be determined using a device and method for determining the parameters set as described further below in more detail.

In some embodiments, the model parameter set includes an auxiliary coefficient set, in particular an auxiliary matrix.

The relaxed patient decomposition coefficient set of such embodiment is determined as a product of a covariance matrix as explained before, and the auxiliary matrix.

In an embodiment, the step of determining the relaxed patient decomposition coefficient set considers a statistic uncertainty of the stressed patient topography data set. The statistic uncertainty may especially result from the measurement uncertainty that is generally present when acquiring the surface topologies, for example with an ophthalmologic measurement device. The statistic uncertainty can be considered as noise and typically follows a normal distribution.

As explained in more detail further below in the context of exemplary embodiments, the statistic uncertainty may also be taken into account when determining the parameters of the statistic model.

In an embodiment, the parametric decompositions are Zernike decompositions and the decomposition coefficients are Zernike coefficients.

Zernike decompositions are well known and particularly useful in the description of optic systems and their properties. Other types of parametric decompositions, however, may be used as well. References to Zernike decompositions, Zernike coefficients etc. in specific examples and exemplary embodiments are accordingly generally to be understood as reference to parametric decomposition.

Further exemplary parametric decompositions that may be used in the present context include Radial Basis Functions (RBFs), Spline-patches, such as Non-Uniform Rational b-Splines (NURBS), or Chebychev Polynomials.

In some embodiments, the step of determining the stressed reference decomposition coefficient set is computed to be centred, thus having a mean of zero for each decomposition coefficient. In an embodiment, the patient tissue includes corneal tissue. In addition to corneal tissue, the patient tissue may include surrounding sclera tissue. In an embodiment, the at least one tissue surface includes an anterior and a posterior corneal surface. As mentioned before, however, other tissues, in particular soft tissues, such as alignments or arteries, may be modelled as well.

In an embodiment, the method further including the step of determining, from relaxed patient topography data set, a relaxed finite element model of the patient tissue. The finite element model is favourably of the same type and computed in the same way as the stressed and reference finite element models that may be in the context of a method for determining a parameter set, as discussed further below. A suited type of finite element model is, for example, disclosed in the EP 2824599 A1.

The relaxed finite element model may serve as starting point for the subsequent planning and/or simulation of surgical interventions, such as ophthalmologic surgical interventions.

In an embodiment, the model parameter set is pre-determined by a computer-implemented method from a stressed reference decomposition coefficient set and a relaxed reference decomposition coefficient set.

According to a further aspect, the overall object is achieved by providing a computerized device for modelling human or animal patient tissue for the simulation of a surgical intervention. The device includes a processor. The processor is configured to control the device to acquire a stressed patient topography data set, the stressed patient topography data set describing a topography of at least one patient tissue surface under mechanical stress. The processor is further configured to control the device to determine, from the stressed patient topography data set, an input data set. The processor is further configured to control the device to evaluate a statistic model for the input data set, thereby obtaining an output data set. The processor is further configured to control the device to determine from the output data set, a relaxed patient topography data set, the relaxed patient topography data set describing a topography of the at least one patient tissue surface in absence of mechanical stress.

The statistic model includes a set of Gaussian Processes and is defined by a pre-determined model parameter set, the model parameter set including at least one Gaussian Process parameter for each Gaussian Process. The statistic model being independent from the patient tissue to be modelled.

In particular embodiments, the processor is configured to control the device to carry out method steps of exemplary methods for modelling human or animal patient tissue as discussed above as well further below in the context of exemplary embodiments. Therefore, the disclosure of such method embodiments is at the same time a disclosure for a corresponding embodiment of a computerized device for modelling human or animal patient tissue.

According to a still further aspect, the overall object is achieved by providing a method for determining a model parameter set for use in the simulation of human or animal patient tissue. The model parameter set including at least one Gaussian Process parameter for each of a set of Gaussian Processes. The method includes the step of acquiring a number of stressed reference topography data sets, each stressed reference topography data set describing a topography of a least one tissue surface of one of a set of reference tissues under mechanical stress. The method further includes the step of determining a parametric decomposition from each of the stressed reference topography data sets, thus obtaining a stressed reference decomposition coefficient set, the stressed reference decomposition coefficient set having a number of stressed reference decomposition coefficients per stressed reference topography data set. The method further includes the step of determining, from the number of stressed reference topography data sets, a corresponding number of stressed reference finite element models. The method further includes determining, from the number of stressed reference finite element models, a corresponding number of relaxed reference finite element models and a corresponding number of relaxed reference topography data sets. The method further includes the step of determining, from each of the relaxed reference topography data sets, a parametric decomposition, thus obtaining a relaxed reference decomposition coefficient set, the relaxed reference decomposition coefficient set having a number of relaxed reference decomposition coefficients per stressed reference topography data set. The method further includes the step of determining, from the stressed reference decomposition coefficient set and the relaxed reference decomposition coefficient set, the at least one Gaussian Process parameter for each of the set of Gaussian Processes. Each Gaussian Process defines a relation between the coefficients of the stressed reference decomposition coefficient set and a corresponding coefficient of the relaxed reference decomposition coefficient set.

The carrying out the method for determining a model data set may also be regarded as a “training” of the statistic model that is subsequently used for the patient tissue modelling, and the model data set may be regarded as “training result”.

The parametric decompositions of the stressed reference topography data sets are of the same type and of the same order, i. e. have the same number of coefficients, as a parametric decomposition that is subsequently used for the patient tissue modelling in clinical application as described above.

In an embodiment, the method includes determining the at least on Gaussian Process Parameter for each Gaussian Process by a numeric fitting and/or optimization process. The fitting may for example be performed by maximizing a marginal likelihood as discussed further below in more detail in the context of exemplary embodiments. The numeric fitting and/or optimization may be done independent for each Gaussian Process.

In an embodiment, the method includes determining and evaluating an auto covariance matrix, wherein the elements of the auto covariance matrix are covariances between all coefficients of stressed reference coefficient sets for all reference tissues. In an embodiment, an auto covariance matrix is determined separately for each of the Gaussian Processes.

According to a still further aspect, the overall object is achieved by providing a computerized device for determining a model parameter set for use in the simulation of human or animal patient tissue. The model parameter set includes at least one Gaussian Process parameter for each of a set of Gaussian Processes. The device includes a processor. The processor is configured to control the device to acquire a number of stressed reference topography data sets, each stressed reference topography data set describing a topography of a least one tissue surface of one of a set of reference tissues under mechanical stress. The processor is further configured to control the device to determine a parametric decomposition from each of the stressed reference topography data sets, thus obtaining a stressed reference decomposition coefficient set, the stressed reference decomposition coefficient set having a number of stressed reference decomposition coefficients per stressed reference topography data set. The processor is further configured to control the device to determine, from the number of stressed reference topography data sets, a corresponding number of stressed reference finite element models. The processor is further configured to control the device to determine, from the number of stressed reference finite element models, a corresponding number of relaxed reference finite element models and a corresponding number of relaxed reference topography data sets. The processor is further configured to control the device to determine, from each of the relaxed reference topography data sets, a relaxed parametric decomposition, thus obtaining a relaxed reference decomposition coefficient set, the relaxed reference decomposition coefficient set having a number of relaxed reference decomposition coefficients per stressed reference topography data set. The processor is further configured to control the device to determine, from the stressed reference decomposition coefficient set and the relaxed reference decomposition coefficient set, at least one Gaussian Process parameter for each of the set of Gaussian Processes. Each Gaussian Process defines a relation between the coefficients of the stressed reference decomposition coefficient set and a corresponding coefficient of the relaxed reference decomposition coefficient set.

In particular embodiments, the processor is configured to control the device to carry out method steps of exemplary methods for determining a model parameter set as discussed above as well further below in the context of exemplary embodiments. Therefore, the disclosure of such method embodiments is at the same time a disclosure for a corresponding embodiment of a computerized device for determining a model parameter set.

According to a still further aspect, the overall objective is achieved by providing a non-transient computer-readable medium with a computer program stored thereon, the computer program being configured to control a processor to execute a method as generally discussed above and/or further below by way of example.

According to a still further aspect, the overall objective is achieved by using a statistic model, the statistic model including a set of Gaussian Processes and being defined by a pre-determined model parameter set, the model parameter set including at least one Gaussian Process parameter for each Gaussian Process and being independent from a patient tissue, for determining at least one relaxed patient tissue surface topography from an acquired stressed patient tissue surface topography.

### BRIEF DESCRIPTION OF THE DRAWINGS

FIG. 1 schematically shows an exemplary computational device;

FIG. 2 schematically shows the corneal and sclera tissue of a human eye as exemplary tissue;

FIG. 3 schematically shows an exemplary operational flow of a method for determining a model parameter set;

FIG. 4 schematically shows an exemplary operational flow of a Gaussian parameter fitting method;

FIG. 5 schematically shows an exemplary operational flow of a method for modelling human or animal tissue;

FIG. 6 schematically shows an exemplary interaction between a computerized device for modelling human or animal tissue and computerized devices for determining a parameter set.

### DETAILED DESCRIPTION OF THE PREFERRED EMBODIMENTS

In the following, exemplary embodiments are discussed with additional reference to the figures.

Throughout this documents, subscript variables in parenthesis “( )” generally refer to index variables.

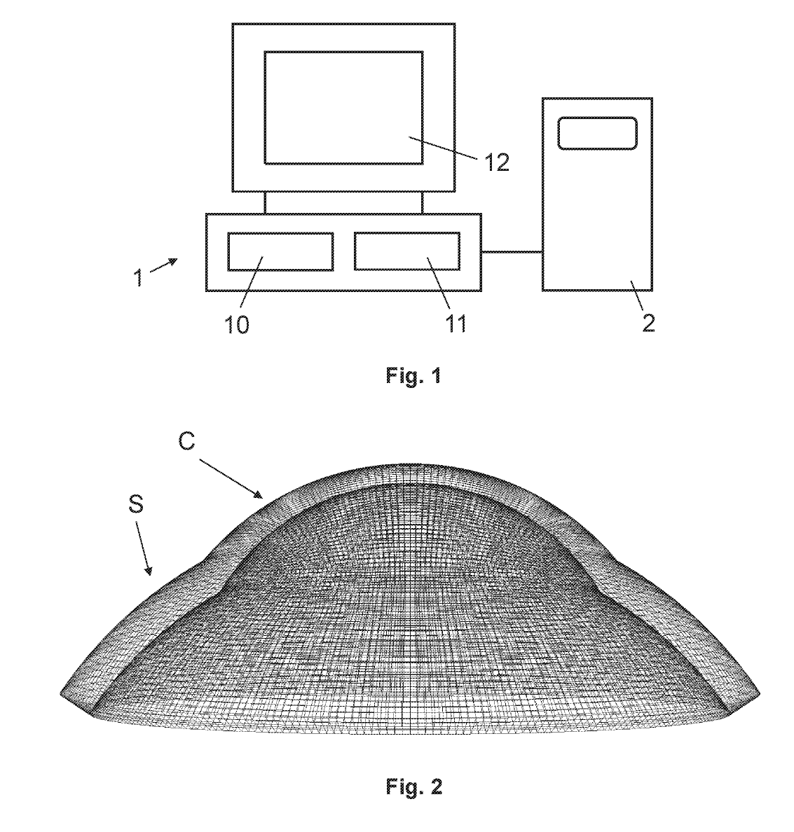

Reference is first made to FIG. 1. FIG. 1 shows a computerized device 1 for modelling human or animal tissue for the simulation of a surgical intervention. In the following, the tissue is exemplarily assumed to be tissue of a human eye and include corneal and sclera tissue.

The device 1 comprises an operational computer with at least one processor 10. The device 1 further comprises a memory 11 in operative coupling with the processor 10 for storing data (data memory) and a computer program code (program memory). In the embodiment of FIG. 1, the device 1 further includes a display 12, such as an LED or LCD screen, a CRT, or any combination thereof. As schematically shown in FIG. 1, the device 1 is, in an embodiment, operatively coupled with an exemplarily computerized and external data source 2, for example via a wired or wireless communication interface. The data source 2 is exemplarily an ophthalmological measurement device for measuring the topography of the anterior and posterior corneal surface, respectively, thus acquiring a (stressed) topography data set of a patient's cornea. Alternatively or additionally to an ophthalmologic measurement device, the data source 2 may be a memory, such as a server or hard disc that stores a single or multiple topography data sets, a CD-ROM, or the like.

The processor 1 is adapted or programmed to control the device 1 to execute method for modelling human or animal tissue for the simulation of a surgical intervention. For this purpose, the device 1 includes a computer program product with a computer-readable medium which is permanently or removable coupled to the processor 10 and comprises a computer program code stored therein. The computer program code is configured to control the processor 10 to execute the method.

Alternatively or additional, the device 1 is a computerized device for determining a parameter set for use in the simulation of human or animal tissue with a corresponding computer program product and computer-readable medium.

In the following, reference is additionally made to FIG. 2. FIG. 2 shows the corneal tissue C and sclera tissue S of a human as exemplary tissue together with a finite element mesh.

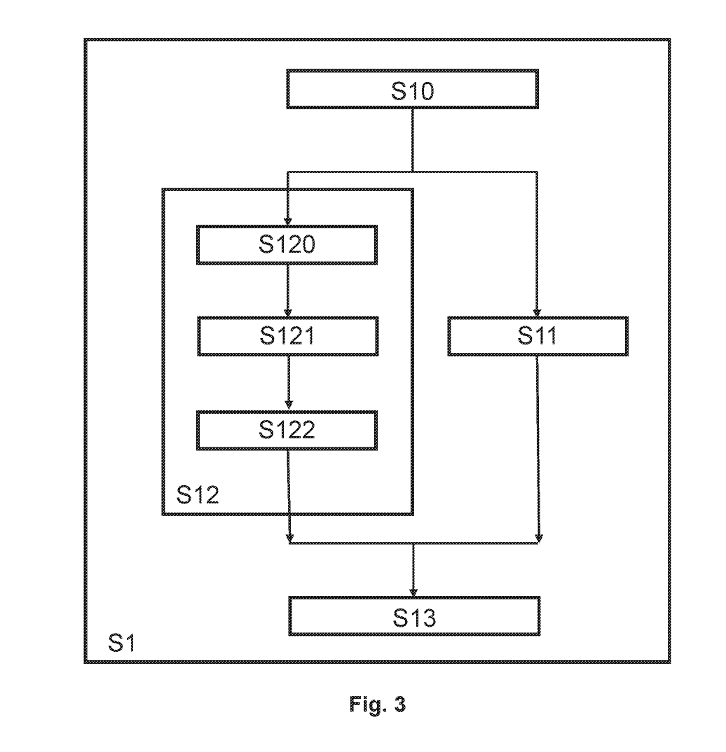

In the following, reference is additionally made to FIG. 3. FIG. 3 schematically shows the operational flow of a method for determining a parameter set for use in the simulation of human or animal tissue, exemplary corneal and sclera tissue as discussed before. In combination, the steps of the method are referred to as S1.

In a first step S10, a number of n stressed reference topography data sets is acquired. The stressed reference topography data sets are exemplarily acquired by in vivo cornea measurements of n reference corneas. The measurements are carried out with the ophthalmologic measurement device 2, from which the data are transmitted to the device 1. For each reference cornea, the topography of the corresponding anterior surface and posterior surface is determined and the corresponding data set exemplarily comprises the topography data of both the anterior and posterior corneal surface. The corresponding surfaces and their topographies are referred to as stressed reference surfaces and stressed reference topographies, respectively.

The stressed reference surface topographies may be determined form healthy and/or from pathological reference corneas. The number of reference corneas may be in a typical range of n≈1000.

Subsequently, the steps S11 and S12 are carried out independently parallel or in sequence.

In the step S11, a Zernike decomposition as exemplary parametric decomposition is determined form each of the n stressed reference topography data sets.

As explained before, each stressed reference topography may be described by the elevation h of each point of the surface over a reference plane. Using cylinder coordinates (ρ, θ), the corresponding Zernike decomposition can be defined as weighted sum of Zernike polynomials:

$$h ( ρ , θ ) = {\sum }_{n = 0}^{k} {\sum }_{m = - n}^{n} {W}_{n}^{m} \cdot {Z}_{n}^{m} ( ρ , θ ) , \qquad ( 1 )$$

with k being the order of the Zernike decomposition, Zₙ^{m}(r, θ) being (tabulated) Zernike polynomials that are favourably stored in the memory 11, and Wₙ^{m} being the Zernike coefficients (weighting factors). In principle, a decomposition up to any desired order k can be computed, wherein the accuracy increases with the order. However, the computational effort both for determining the Zernike decomposition as well as for the subsequent steps as will be discussed below increases with the order. A reasonable order for practical purposes, is, e.g. k=6, i.e. a 6^{th} order Zernike decomposition. The number of non-zero Zernike coefficients is given by

$$\frac{k \cdot ( k + 3 )}{2} + 1.$$

For a Zernike decomposition of order k=6, 28 Zernike coefficients are accordingly computed for each of the anterior and the posterior tissue surface.

It is noted that a double indexing scheme is used for the Zernike coefficients Wₙ^{m}, as common in the context of Zernike decompositions. For practical computational purposes, however, a single indexing scheme for all Zernike coefficients is favourable and is used in the following. A table of Zernike coefficients up to the 7-th order is provided in Table A, together with a single-indexing scheme with index variable j.

In step S11, the Zernike decomposition is determined for each of the stressed reference topography data sets by evaluating Eqn (1) applying algorithms as generally known in the art, for example a fitting algorithm, such a least square fitting algorithm. Thereby, a set of Zernike coefficients is determined for each corneal surface that best describes the surface topography over the whole cornea.

Further in step S11, the Zernike coefficients of the single stressed reference surfaces are combined to a stressed reference Zernike coefficient set. The stressed reference Zernike coefficient set is favourably arranged as an n×m-Matrix X_{R} with a row for each of the n reference corneas and a column for each of the m Zernike coefficients of the single Zernike decompositions. The Matrix X_{R} is referred to as stressed reference Zernike coefficient matrix and is stored in the memory 11. Generally, a set of all Zernike coefficients for a specific cornea (e.g. a specific row of the stressed reference Zernike coefficient matrix X_{R}) is also referred to as Zernike coefficient vector for the cornea. The stressed reference Zernike coefficient Matrix X_{R} forms part of the model parameter set that is subsequently used for the modelling of patient corneas.

As noted before, two sets of Zernike coefficients are determined for each reference cornea, namely for the anterior corneal surface and the posterior corneal surface, respectively. In an exemplary implementation that is assumed in the following, the Zernike coefficients for the anterior and the posterior corneal surface are arranged in a common stressed reference Zernike coefficient matrix X_{R}, with, e.g., the first half of columns being associated with the anterior corneal surfaces and the second half of columns being associated with the posterior corneal surfaces. Further arrangements, however, may be used as well. Furthermore, the Zernike coefficients that are associated with the anterior surfaces and posterior surfaces, respectively, may be arranged in separate matrices.

The Step S12 comprises the steps S120, S121, S122. In the step S120, a number of n stressed reference finite element models is determined from the n stressed reference topography data sets. Different types of finite element models that may be adapted for modelling a specific human or animal tissue are known in the art for a variety of purposes. A type of model that is particularly suited in the context of ophthalmology is disclosed in the EP 2824599 A1 to which reference is herewith made regarding a suited type of finite element model. For the exemplary case of corneal tissue and sclera tissue, a spherical finite element mesh as shown in FIG. 2 may be used.

In the subsequent step S121, a number of n relaxed reference finite element models is determined from the n stressed reference finite element models, assuming a physiological intraocular pressure in a range, of 12 mmHg to 19 mmHg, e.g. 15 mmHg.

In the method that is applied for determining the relaxed (reference) finite element models, the nodes of the finite element mesh of the stressed (reference) finite element models are progressively moved towards the relaxed configuration in an iterative process as described by Pandolfi (Pandolfi, A., Holzapfel, G. A., 2008. Three-dimensional modeling and computational analysis of the human cornea considering distributed collagen fibril orientations. Journal of Biomechanical Engineering 130, (6):061006) and by Elsheikh (Elsheikh, A., Whitford, C., Hamarashid, R., Kassem, W., Joda, A., Büchler, P., (2012). Stress free configuration of the human eye. Medical Engineering and Physics 35, 211-216), to which reference is herewith made regarding the determination of the relaxed finite element models. However, alternative and e. g. patient-specific models may be used as well

The anterior respectively posterior surface of the determined relaxed reference finite element models form relaxed reference surfaces with associated relaxed reference topographies.

In the subsequent step S122, a Zernike decomposition is determined form each of the relaxed reference finite element models in an analogue way as described before with reference to step S11 and the stressed reference surfaces. As result of the step S122, a relaxed reference Zernike coefficient set is provided. The relaxed reference Zernike coefficient set is favourably arranged in the same way as the stressed reference Zernike coefficient set X_{R}, particularly as an n×m-Matrix Y_{R}. Further in the step S122, the relaxed Zernike coefficient set Y_{R} is favourably centred by subtracting, in each column, (corresponding to a specific Zernike coefficient for all reference corneas), the mean of the column from all elements.

In the subsequent step S13, Gaussian Process parameters of a set of m Gaussian Process are determined from the stressed reference Zernike coefficient set X_{R} and the relaxed reference Zernike coefficient set Y_{R}.

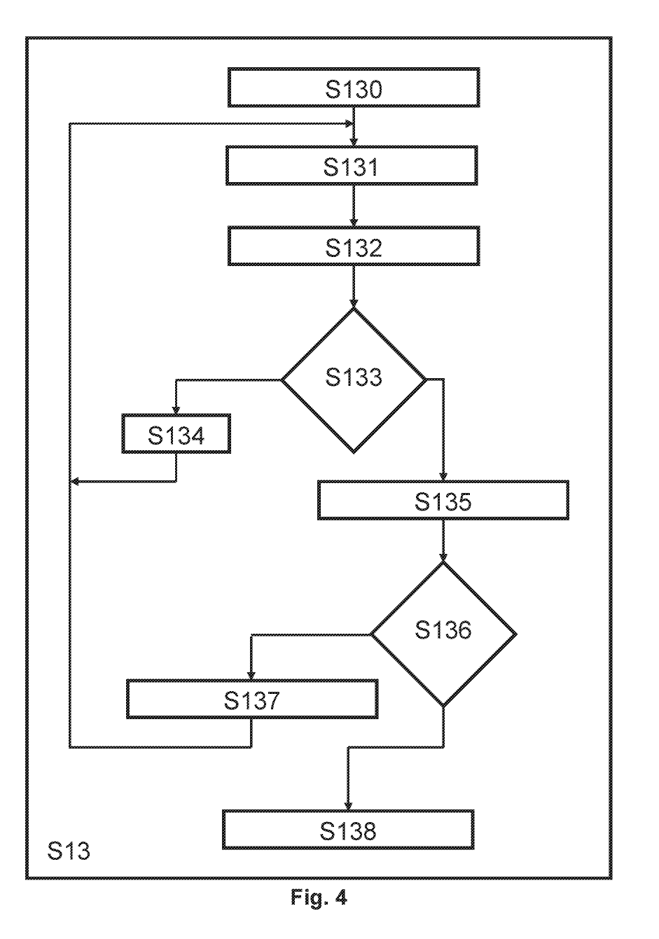

In the following, reference is additionally made to FIG. 4. FIG. 4 shows an exemplary implementation of the step S13 of FIG. 3 in a more detailed view.

In the step S130, a reference auto distance matrix D_{A} is computed that will be used in subsequent computational steps. The reference auto distance matrix is exemplarily computed as follows:

With A, B being matrixes of n_{A} respectively n_{B} rows and an identical number of m columns, and with a_{(i,j)}), b_{(i,j)} being corresponding matrix elements, an element d_{(i,j)} of a corresponding distance matrix D(A, B) with dimension n_{A}×n_{B} can be computed as

$${d}_{( i , j )} = {\sum }_{k = 1}^{m} {( {a}_{( i , k )} - {b}_{( j , k )} )}^{2} , \qquad ( 2 )$$

with i running from 1 to n_{A} and j running from 1 to n_{B}. From Eqn (2) it can directly be seen that the distance matrix D is symmetrical which is favourably exploited for an efficient computation.

The reference auto distance matrix D_{A} is a obtained as

D_{A}=D(X_{R}, X_{R}) (2a).

The reference auto distance matrix D_{A} comprises the squared distances of the stressed reference Zernike coefficient vector (rows of the stressed reference Zernike coefficient matrix X_{R}) for each reference cornea to the stressed reference coefficient vectors of all other reference corneas. Besides from being symmetrical, it directly follows from Eqn (2) that the elements of the principal diagonal of the reference auto distance matrix D_{A} are zero, which is favourably also exploited for an efficient computation.

In the subsequent steps, m triples of Gaussian Process parameters (σ, l, σ_{N}) are determined for m Gaussian Processes, each Gaussian Process being associated with a specific Zernike coefficient.

With (σ², l) being a tuple of scalar Gaussian Process parameters and with A, B, D being matrices as defined above, the elements k_{(i,j)} of a covariance matrix K with the same dimensions as the distance matrix D can be computed as

k_{i,j}=σ²·e^{d}^{_{i,j}}^{/(2l)}^{²} (3)

for i running from 1 to n_{A} and j running from 1 to n_{B}. The covariance matrix K can be expressed as a function of the matrices A and B, i. e. K=K(A, B), or—equivalently, as function of the distance matrix D, i. e. K=K(D), with (σ², l) as given parameters of the covariance matrix K, wherein σ² is a variance and l is a scaling factor.

A covariance matrix K as explained before is the covariance matrix of a Gaussian Process, the Gaussian Process being fully defined by its mean and covariance functions.

Since the data are in this exemplary embodiment, are centred as explained before, the mean m(x) is a zero vector and the Gaussian Process is defined by the covariance matrix K in dependence of the tuple of Gaussian Process parameters (σ², l). In alternative embodiments where the data are not cantered, the Gaussian process parameters further include the mean.

For each of the m Gaussian Processes, or—equivalently—for each of the m Zernike coefficients in each Zernike coefficient vector, the computation of the Gaussian Process parameter triple (σ, l, σ_{N}) is carried out in a fitting process, with σ_{N(r)} being an optional noise variance as explained further below. The computation is carried out independently favourably sequentially all m Gaussian Processes. In the following, σ₍ᵣ₎, l₍ᵣ₎, σ_{N(r)} refer to the r-th set of Gaussian Process parameter, or Gaussian Process parameter triple, with r being an index parameter running from 1 to m. Each Gaussian Process Parameter triple (σ₍ᵣ₎, l₍ᵣ₎, σ_{N(r)}) may be considered as Gaussian Process parameter tuple (σ₍ᵣ₎, l₍ᵣ₎) as explained before, and σ_{N(r)}² as additional noise variance. In case no noise is considered, the triples (σ₍ᵣ₎, l₍ᵣ₎, σ_{N(r)}) are replaced by the tuples (σ₍ᵣ₎, l₍ᵣ₎).

The computation starts with r=1, i.e for the first Zernike coefficient with starting points or initial values for σ₍ᵣ₎, l₍ᵣ₎, σ_{N(r)}.

In the step S131, a logarithmic marginal likelihood log(p) is computed via

$$log ( p ) = - \frac{1}{2} \cdot {y}_{( r )}^{T} \cdot {C}^{- 1} \cdot {y}_{( r )} - \frac{1}{2} \cdot log ( \vert C \vert ) - \frac{n}{2} \cdot log ( 2 π ) , \qquad ( 5 )$$

with y₍ᵣ₎ being a vector (i. e. a matrix of dimension n×1) with the r-th relaxed reference Zernike coefficient for all reference corneas. C is a matrix of dimension n×n that is commuted as

C=K(D_{A})+σ_{N(r)}²·I (5a).

In Eqn (5a), K(D_{A}) is an auto covariance matrix, with the elements being computed according Eqn (3) with σ=σ₍ᵣ₎ and l=l₍ᵣ₎.

Further in Eqn (5a), σ_{N(r)}² is—for each Zernike coefficient—a noise variance that characterizes the statistic uncertainty of the variables of the Zernike coefficients as variables of the Gaussian Process. This noise includes the uncertainty of the surface topography measurements, as well as the uncertainty of the computed relaxed reference Zernike coefficient set Y_{R}. Hence, the noise is related to the statistic uncertainty of the training data. The statistic uncertainty is exemplarily assumed as being normally distributed, which is typically the case. Further in Eqn (5a), I is a unit matrix. The noise variance σ_{N(r)}² is determined as part of the fitting/optimization process. It is noted that considering the statistic uncertainty is favourable but not essential.

In the three summands of Eqn (5), the first term is a fitting term as such, the second term is a penalty term and the third term is a normalizing constant.

In combination, all m Gaussian Process parameter triples (σ², l, σ_{N}²) form a hyper parameter θ that is exemplary arranged and stored as Gaussian Process parameter matrix of order m×3. The Gaussian Process parameter matrix θ forms part of the model parameter set that is subsequently used for the modelling of patient corneas.

In the subsequent step S132, it is assessed whether the marginal likelihood p (in strictly monotonous relationship with its logarithm log(p)) has assumed a maximum for the current values for the Gaussian Process parameter triple (σ₍ᵣ₎, l₍ᵣ₎, σ_{N(r)}) and the operational flow branches in the subsequent step S133. In the negative, case, i. e. if no maximum has been established, the Gaussian Process parameter triple (σ₍ᵣ₎, l₍ᵣ₎, σ_{N(r)}) is modified in the step S134 and the operational flow proceed with step S131.

In the affirmative case, i. e. if a maximum has been established, the operational flow proceeds with the step S135.

The steps S131, S132, S133, S134, in combination, are a simplified representation of a numerical fitting or maximization (optimization) algorithm for the marginal likelihood p. The detailed implementation may be carried out according to established algorithms that are known in the art. Exemplary algorithms that may be used include BFGS (Broyden, Fletcher, Goldfarb-Shanno); LBFGS (Limited memory—BFGS) or CG (Conjugate Gradient) algorithms.

In the step S135, the computed final values Gaussian Process parameter triple (σ₍ᵣ₎, l₍ᵣ₎, σ_{N(r)}) are stored in the r-th row of the Gaussian Process parameter matrix θ (not explicitly shown).

Further in the step S135, an auxiliary vector

α₍ᵣ₎=C⁻¹·y₍ᵣ₎ (6)

is computed and stored for subsequent use. The m auxiliary vectors α₍ᵣ₎ are favourably arranged as auxiliary matrix A. The auxiliary matrix A forms part of the model parameter set that is subsequently used for the modelling of patient corneas.

In the step S136, it is determined whether the fitting has been carried out for all m Gaussian Processes, i. e. whether r=m. In negative case, the index variable r is incremented by one and starting points are set for the new Gaussian Process parameter triple (σ₍ᵣ₎, l₍ᵣ₎, σ_{N(r)}) in the step S137. Then, the operational flow subsequently proceeds with step S131 as discussed before for fitting the next parameter triple for the next Gaussian Process.

In the affirmative case, the operational flow terminates with the step S138.

In combination, the m Gaussian Processes establish a statistic model for the relation between the stressed and the relaxed reference Zernike coefficients, respectively.

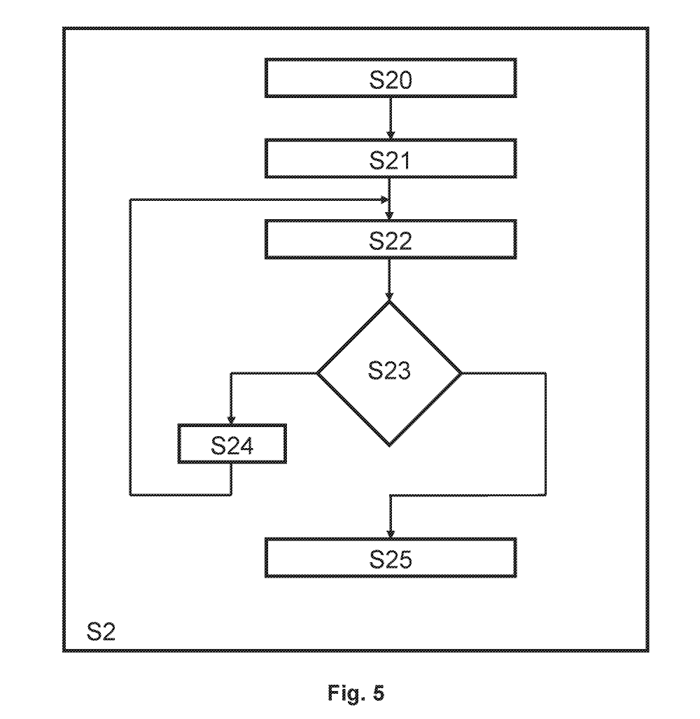

In the following, reference is additionally made to FIG. 5. FIG. 5 schematically shows the operational flow of a method for modelling human or animal tissue for the simulation of a surgical intervention. In combination, the steps of the method are referred to as S2.

In the following, it is assumed that the method is carried out with a device as generally discussed before with reference to FIG. 1. It is further assumed that a parameter set as discussed before and that a model parameter set that includes the stressed reference Zernike coefficient matrix X_{R}, the Gaussian Process parameter matrix θ, and the auxiliary matrix A are stored in the memory 11, e.g. as part of the program code, or are otherwise available for the computation.

In the step S20, a stressed patient topography data set of the anterior and posterior surface of a patient's corona is acquired, e. g. by in vivo measurement with the ophthalmologic measurement device 2, or such stressed patient topography data set is red in from a data source.

In the subsequent step S21, a Zernike decomposition is determined form the stressed patient topography data set in an analogue way as discussed before with reference to Eqn 1. The computed m Zernike coefficients are stored in a stressed patient Zernike coefficient vector X_{P}.

In the subsequent steps, a relaxed patient Zernike coefficient vector Y_{P} is determined is determined as follows. The computation is exemplarily carried out in a loop for one coefficient after the other, with an index variable r running from 1 to m.

In the step S22, the r-th relaxed patient Zernike coefficient y_{P(r)} is computed by.

y_{P(r)}=K(X_{P}, X_{R})·α₍ᵣ₎ (7).

In Eqn (7), K(X_{P}, X_{R}) is an (1×n) cross covariance matrix (i. e. a vector) for the covariances between the stressed reference Zernike coefficient matrix X_{R} and the stressed patient Zernike coefficient vector X_{P}. The computation of Eqn (7) is carried out with the r-th Gaussian Process parameter triple (σ₍ᵣ₎, l₍ᵣ₎, σ_{N(r)}).

The right side of Eqn 7 corresponds to the expected value for the r-th Gaussian Process as determined before with X_{P} as argument and is given by a linear combination of the covariance matrix K(X_{P}, X_{R}) and the pre-computed vector α₍ᵣ₎.

It is noted that Eqn (7) can be computed efficiently and in a straight forward way, without requiring computational expensive operations and particularly without requiring the computation of a relaxed finite element model from a stressed finite element model. Using, e.g. a state-of the-art personal computer (PC) or workstation, the relaxed surface topography is computed in a range of seconds or even fractions of seconds.

In the subsequent step S23, it is determined whether all elements of the relaxed patient Zernike coefficient vector Y_{P} have been determined, i. e. whether r=m. In the negative case the index variable r is increased by one and the operational flow proceeds with the step S22 for the next coefficient.

In the affirmative case, the operational flow proceeds with the step S25. In the step S25, a relaxed patient topography data set of the patient's anterior and posterior corneal surface is determined based on Eqn (1) and the relaxed patient Zernike coefficient vector Y_{P} as previously determined

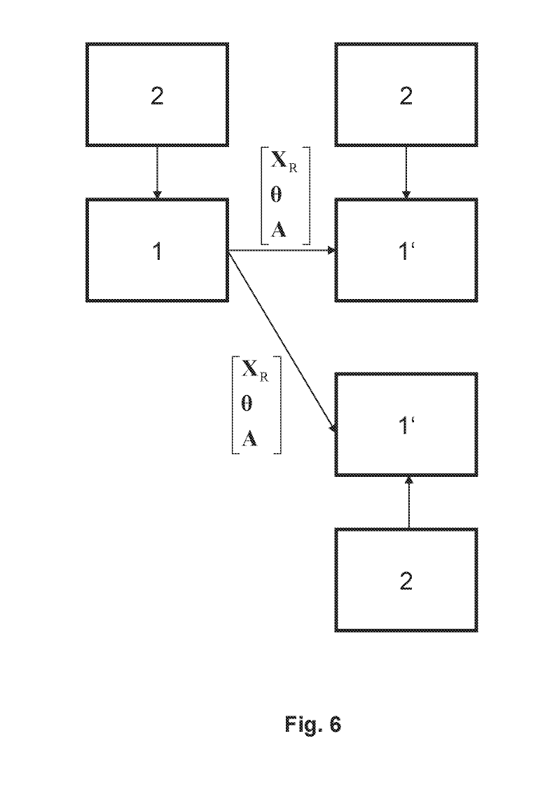

In the following, reference is additionally made to FIG. 6. FIG. 6 schematically shows an exemplary interaction between computerized devices 1′ for determining a model parameter set and a device 1 for modelling human or animal tissue. The tissue is again exemplarily assumed to be eye tissue, and include corneal tissue. Both the devices 1 and 1′ respectively, may be designed as discussed above with reference to FIG. 1.

The device 1 carries out a method for determining a model parameter set as described above with particular reference to FIGS. 3, 4, thereby determining the stressed reference Zernike coefficient matrix X_{R}, the Gaussian Process parameter matrix θ and the auxiliary matrix A as described before, which form, in combination a model parameter set. Since this method involves complex computations and in particular comprises, for each reference cornea, the determination of an unstressed finite element model from a stressed finite element model as described before, the required computational effort and the computation time are considerable.

The determined model parameter set is subsequently transferred to device 1′ for modelling tissue. The transfer of the model data set may be carried out in different ways, e. g. via a wired or wireless communication interface, or via a parameter or coefficient file that is read by the devices 1′ or as part of a program code that is running on the devices 1′. Furthermore, the device 1′ may be structurally integral with the device 1 and be realized, e.g. by a common workstation. In this context, it is only required that the model data set is available on the devices 1′ for subsequent use.

As described before, determining of the relaxed surface topography can be carried out quickly and efficiently based on Eqn (7), once the model parameter set is given. Therefore, device 1′ can be used and the method for modelling patient tissue can be used efficiently in the clinical routine applications.

It is to be further understood that the number two devices 1′ is merely exemplary. The model data set may be distributed to any desired number of devices 1′ and/or copied to any desired number of parameter or coefficient file, and/or be included in any desired number of program codes.

For the methods and devices as described before, the description of the operational flow and the single method steps are described in a manner that best aids the reader's understanding. It is to be understood, however, that the methods may be implemented in somewhat different ways. In particular, single method steps may be split into a number of sub steps, while separately described steps may be combined. Furthermore, the computational order of single steps may be changed or modified. The arrangement of data in variables, lists, matrices, vectors and the like may be carried out under memory and computational efficiency aspects as know in the art. Also, the reference to specific numerical algorithms, such as fitting or optimization algorithms, mean and covariance functions, likelihood functions etc. is to be understood merely exemplary and not limited to any specific implementation and/or formula. Other algorithms and formulas as known in the art may be used as well.

TABLE A
Zernike polynomials (as published in WO 2008077006 A1)
Table 1. Listing of Zernike Polynomials in Polar Coordinates up to
7^{th} order (36 terms).

| j = | | | |
|---|---|---|---|
| index | n = order | m = frequency | Zₙ^{m} (ρ, θ) |
| 0 | 0 | 0 | 1 |
| 1 | 1 | −1 | 2 ρ sin θ |
| 2 | 1 | 1 | 2 ρ cos θ |
| 3 | 2 | −2 | \sqrt{6} ρ² sin 2θ |
| 4 | 2 | 0 | \sqrt{3} (2ρ² − 1) |
| 5 | 2 | 2 | \sqrt{6} ρ² cos 2θ |
| 6 | 3 | −3 | \sqrt{8} ρ³ sin 3θ |
| 7 | 3 | −1 | \sqrt{8} (3ρ³ − 2ρ) sin θ |
| 8 | 3 | 1 | \sqrt{8} (3ρ³ − 2ρ) cos θ |
| 9 | 3 | 3 | \sqrt{8} ρ³ cos 3θ |
| 10 | 4 | −4 | \sqrt{10} ρ⁴ sin 4θ |
| 11 | 4 | −2 | \sqrt{10} (4ρ⁴ − 3ρ²) sin 2θ |
| 12 | 4 | 0 | \sqrt{5} (6ρ⁴ − 6ρ² + 1) |
| 13 | 4 | 2 | \sqrt{10} (4ρ⁴ − 3ρ²) cos 2θ |
| 14 | 4 | 4 | \sqrt{10} ρ⁴ cos 4θ |
| 15 | 5 | −5 | \sqrt{12} ρ⁵ sin 5θ |
| 16 | 5 | −3 | \sqrt{12} (5ρ⁵ − 4ρ³) sin 3θ |
| 17 | 5 | −1 | \sqrt{12} (10ρ⁵ − 12ρ³ + 3ρ) sin θ |
| 18 | 5 | 1 | \sqrt{12} (10ρ⁵ − 12ρ³ + 3ρ) cos θ |
| 19 | 5 | 3 | \sqrt{12} (5ρ⁵ − 4ρ³) cos 3θ |
| 20 | 5 | 5 | \sqrt{12} ρ⁵ cos 5θ |
| 21 | 6 | −6 | \sqrt{14} ρ⁶ sin 6θ |
| 22 | 6 | −4 | \sqrt{14} (6ρ⁶ − 5ρ⁴) sin 4θ |
| 23 | 6 | −2 | \sqrt{14} (15ρ⁶ − 20ρ⁴ + 6ρ²) sin 2θ |
| 24 | 6 | 0 | \sqrt{7} (20ρ⁶ − 30ρ⁴ + 12ρ² − 1) |
| 25 | 6 | 2 | \sqrt{14} (15ρ⁶ − 20ρ⁴ + 6ρ²) cos 2θ |
| 26 | 6 | 4 | \sqrt{14} (6ρ⁶ − 5ρ⁴) cos 4θ |
| 27 | 6 | 6 | \sqrt{14} ρ⁶ cos 6θ |
| 28 | 7 | −7 | 4 ρ⁷ sin 7θ |
| 29 | 7 | −5 | 4 (7ρ⁷ − 6ρ⁵) sin 5θ |
| 30 | 7 | −3 | 4 (21ρ⁷ − 30ρ⁵ + 10ρ³) sin 3θ |
| 31 | 7 | −1 | 4 (35ρ⁷ − 60ρ⁵ + 30ρ³ − 4ρ) sin θ |
| 32 | 7 | 1 | 4 (35ρ⁷ − 60ρ⁵ + 30ρ³ − 4ρ) cos θ |
| 33 | 7 | 3 | 4 (21ρ⁷ − 30ρ⁵ + 10ρ³) cos 3θ |
| 34 | 7 | 5 | 4 (7ρ⁷ − 6ρ⁵) cos 5θ |
| 35 | 7 | 7 | 4 ρ⁷ cos 7θ |

1. Computer-implemented method for modelling human or animal patient tissue for the simulation of a surgical intervention, the method including:
acquiring a stressed patient topography data set, the stressed patient topography data set describing a topography of at least one patient tissue surface under mechanical stress,
determining, from the stressed patient topography data set, an input data set;
evaluating a statistic model for the input data set, thereby obtaining an output data set;
determining, from the output data set, a relaxed patient topography data set, the relaxed patient topography data set describing a topography of the at least one patient tissue surface in absence of mechanical stress,
wherein the statistic model includes a set of Gaussian Processes and is defined by a pre-determined model parameter set, the model parameter set including at least one Gaussian Process parameter for each Gaussian Process, and wherein the statistic model is independent from the patient tissue to be modelled.

2. Method according to claim 1,
wherein the input data set is a stressed patient decomposition coefficient set, the stressed patient decomposition coefficient set being obtained via a parametric decomposition from the stressed patient topography data set, the stressed patient decomposition coefficient set forming the input data set;
and wherein the output data set is a relaxed patient decomposition coefficient set,
with the statistic model defining a relation between coefficients of the stressed patient decomposition coefficient set and the relaxed patient decomposition coefficient set.

3. Method according to claim 2, wherein the step of determining the relaxed patient decomposition coefficient set includes evaluating a relation between the coefficients of the stressed patient decomposition coefficient set, the coefficients of a pre-determined stressed reference decomposition coefficient set and the coefficients of a pre-determined relaxed reference decomposition coefficient set.

4. Method according to claim 3, wherein the step of determining the relaxed patient decomposition coefficient set includes evaluating a set of covariances between coefficients of the stressed patient decomposition coefficient set and coefficients of the stressed reference decomposition coefficient set.

5. Method according to claim 2, wherein the parametric decompositions are Zernike decompositions and the decomposition coefficients are Zernike coefficients.

6. Method according to claim 1, wherein the at least one Gaussian Process parameter includes, for each Gaussian Process, a tuple of a variance parameter and a scaling parameter.

7. Method according to claim 1, wherein the step of determining the relaxed patient decomposition coefficient set considers a statistic uncertainty of the stressed patient topography data set.

8. Method according to claim 1, wherein the patient tissue includes corneal tissue.

9. Method according to claim 8, wherein the at least one tissue surface includes an anterior and a posterior corneal surface.

10. Method according to claim 1, the method further including the steps of determining, from the relaxed patient topography data set, a relaxed finite element model of the patient tissue.

11. Method according to claim 1, wherein the model parameter set is pre-determined by a computer-implemented method for determining a model parameter set for use in the simulation of human or animal patient tissue, the model parameter set including at least one Gaussian Process parameter for each of a set of Gaussian Processes, the method including the steps of:
acquiring a number of stressed reference topography data sets, each stressed reference topography data set describing a topography of at least one tissue surface of one of a set of reference tissues under mechanical stress;
determining a parametric decomposition from each of the stressed reference topography data sets, thus obtaining a stressed reference decomposition coefficient set, the stressed reference decomposition coefficient set having a number of stressed reference decomposition coefficients per stressed reference topography data set;
determining, from the number of stressed reference topography data sets, a corresponding number of stressed reference finite element models;
determining, from the number of stressed reference finite element models, a corresponding number of relaxed reference finite element models and a corresponding number of relaxed reference topography data sets;
determining, from each of the relaxed reference topography data sets, a parametric decomposition, thus obtaining a relaxed reference decomposition coefficient set, the relaxed reference decomposition coefficient set having a number of relaxed reference decomposition coefficients per stressed reference topography data set;
determining, from the stressed reference decomposition coefficient set and the relaxed reference decomposition coefficient set, the at least one Gaussian Process parameter for each of the set of Gaussian Processes,
wherein each Gaussian Process defines a relation between the coefficients of the stressed reference decomposition coefficient set and a corresponding coefficient of the relaxed reference decomposition coefficient set.

12. Computerized device for modelling human or animal patient tissue for the simulation of a surgical intervention, the device including a processor, the processor being configured to control the device;
to acquire a stressed patient topography data set, the stressed patient topography data set describing a topography of at least one patient tissue surface under mechanical stress,
to determine, from the stressed patient topography data set, an input data set;
to evaluate a statistic model for the input data set, thereby obtaining an output data set;
to determine, from the output data set, a relaxed patient topography data set, the relaxed patient topography data set describing a topography of the at least one patient tissue surface in absence of mechanical stress,
wherein the statistic model includes a set of Gaussian Processes and is defined by a pre-determined model parameter set, the model parameter set including at least one Gaussian Process parameter for each Gaussian Process, and wherein the statistic model is independent from the patient tissue to be modelled.

13. Computer-implemented method for determining a model parameter set for use in the simulation of human or animal patient tissue, the model parameter set including at least one Gaussian Process parameter for each of a set of Gaussian Processes, the method including the steps of:
acquiring a number of stressed reference topography data sets, each stressed reference topography data set describing a topography of a least one tissue surface of one of a set of reference tissues under mechanical stress;
determining a parametric decomposition from each of the stressed reference topography data sets, thus obtaining a stressed reference decomposition coefficient set, the stressed reference decomposition coefficient set having a number of stressed reference decomposition coefficients per stressed reference topography data set;
determining, from the number of stressed reference topography data sets, a corresponding number of stressed reference finite element models;
determining, from the number of stressed reference finite element models, a corresponding number of relaxed reference finite element models and a corresponding number of relaxed reference topography data sets;
determining, from each of the relaxed reference topography data sets, a parametric decomposition, thus obtaining a relaxed reference decomposition coefficient set, the relaxed reference decomposition coefficient set having a number of relaxed reference decomposition coefficients per stressed reference topography data set;
determining, from the stressed reference decomposition coefficient set and the relaxed reference decomposition coefficient set, the at least one Gaussian Process parameter for each of the set of Gaussian Processes,
wherein each Gaussian Process defines a relation between the coefficients of the stressed reference decomposition coefficient set and a corresponding coefficient of the relaxed reference decomposition coefficient set.

14. Method according to claim 13, the method including determining the at least one Gaussian Process Parameter for each Gaussian Process by a numeric fitting and/or optimization process.

15. Computerized device for determining a model parameter set parameter set for use in the simulation of human or animal patient tissue, the model parameter set including at least one Gaussian Process parameter for each of a set of Gaussian Processes, the device including a processor, the processor being configured to control the device:
to acquire a number of stressed reference topography data sets, each stressed reference topography data set describing a topography of a least one tissue surface of one of a set of reference tissues under mechanical stress;
to determine a parametric decomposition from each of the stressed reference topography data sets, thus obtaining a stressed reference decomposition coefficient set, the stressed reference decomposition coefficient set having a number of stressed reference decomposition coefficients per stressed reference topography data set;
to determine, from the number of stressed reference topography data sets, a corresponding number of stressed reference finite element models;
to determine, from the number of stressed reference finite element models, a corresponding number of relaxed reference finite element models and a corresponding number of relaxed reference topography data sets;
to determine, from each of the relaxed reference topography data sets, a parametric decomposition, thus obtaining a relaxed reference decomposition coefficient set, the relaxed reference decomposition coefficient set having a number of relaxed reference decomposition coefficients per stressed reference topography data set;
to determine, from the stressed reference decomposition coefficient set and the relaxed reference decomposition coefficient set, the at least one Gaussian Process parameter for each of the set of Gaussian Processes,
wherein each Gaussian Process defines a relation between the coefficients of the stressed reference decomposition coefficient set and a corresponding coefficient of the relaxed reference decomposition coefficient set.

16. Non-transient computer-readable medium with a computer program stored thereon, the computer program being configured to control a processor to execute a method according to claim 1.

17. Use of a statistic model, the statistic model including a set of Gaussian Processes and being defined by a pre-determined model parameter set, the model parameter set including at least one Gaussian Process parameter for each Gaussian Process and being independent from a patient tissue, for determining at least one relaxed patient tissue surface topography from an acquired stressed patient tissue surface topography.
